(19) United States
(12) Patent Application Publication
(10) Pub. No.: US 2011/0224471 A1
(43) Pub. Date: Sep. 15, 2011

(54) PROCESS OF CRACKING BIOFEEDS USING HIGH ZEOLITE TO MATRIX SURFACE AREA CATALYSTS

(76) Inventors: Richard Franklin Wormsbecher, Dayton, MD (US); Kevin John Sutovich, Hampstead, MD (US)

(21) Appl. No.: 13/128,139

(22) PCT Filed: Dec. 8, 2009
(86) PCT No.: PCT/US09/06429

§ 371 Date: May 6, 2011

Related U.S. Application Data
(60) Provisional application No. 61/201,198, filed on Dec. 8, 2008.

(51) Int. Cl. C07C 4/00

(52) U.S. Cl. 585/899

(57) ABSTRACT

A process for fluid catalytically cracking a hydrocarbon feedstock containing at least one bio-renewable feed fraction using a rare earth metal oxide-containing, high zeolite-to-matrix surface area ratio catalyst is disclosed. The catalyst comprising a zeolite, preferably a Y-type zeolite, a matrix, at least 1 wt % of a rare earth metal oxide, based on the total weight of the catalyst. The zeolite surface area-to-matrix surface area ratio of the catalyst is at least 2, preferably greater than 2.

### FIELD OF THE INVENTION

The present invention relates to the catalytic conversion of a feedstock containing a bio-renewable feed. More specifically, the present invention relates to a process for fluid catalytically cracking a feedstock containing a bio-renewable feed using a rare earth containing catalytic cracking catalyst having a specified ratio of zeolite-to-matrix surface area.

### BACKGROUND OF THE INVENTION

Fluidized catalytic cracking (FCC) units are used in the petroleum industry to convert high boiling petroleum based hydrocarbon feedstocks to more valuable hydrocarbon products, such as gasoline, having a lower average molecular weight and a lower average boiling point than the feedstocks from which they are derived. The conversion is normally accomplished by contacting the hydrocarbon feedstock with a moving bed of catalyst particles at temperatures ranging between about 427° C. and about 593° C. The most typical hydrocarbon feedstock treated in FCC units is petroleum based and comprises a heavy gas oil, but on occasion, such feedstocks as light gas oils or atmospheric gas oils, naphthas, reduced crudes and even whole crudes are subjected to catalytic cracking to yield low boiling hydrocarbon products.

Catalytic cracking in FCC units generally comprises a cyclic process involving a separate zone for catalytic reaction, steam stripping and catalyst regeneration. The higher molecular hydrocarbon feedstock is converted into gaseous, lower boiling hydrocarbons. Afterward these gaseous, lower boiling hydrocarbons are separated from the catalyst in a suitable separator, such as a cyclone separator, and the catalyst, now deactivated by coke deposited upon its surfaces, is passed to a stripper. The deactivated catalyst is contacted with steam to remove entrained hydrocarbons that are then combined with vapors exiting the cyclone separator to form a mixture that is subsequently passed downstream to other facilities for further treatment. The coke-containing catalyst particles recovered from the stripper are introduced into a regenerator, normally a fluidized bed regenerator, where the catalyst is reactivated by combusting the coke in the presence of an oxygen-containing gas, such as air.

FCC catalysts normally consist of a range of extremely small spherical particles. Commercial grades normally have average particle sizes ranging from about 50 to 150 μm, preferably from about 50 to about 100 μm. The cracking catalysts are comprised of a number of components, each of which is designed to enhance the overall performance of the catalyst. Some of the components influence activity and selectivity while others affect the integrity and retention properties of the catalyst particles. FCC catalysts are generally composed of zeolite, active matrix, clay and binder with all of the components incorporated into a single particle or are comprised of blends of individual particles having different functions.

Bottoms upgrading capability is an important characteristic of an FCC catalyst. Improved bottoms conversion can significantly improve the economics of an FCC process by converting more of the undesired heavy products into more desirable products such as light cycle oil, gasoline and olefins. Bottoms conversion is typically defined as the residual fraction boiling over 343° C. It is desirable to minimize the bottoms yields at constant coke.

In recent years, increased attention has been given to the use of bio-renewable materials as a fuel source. FCC has been reported as one process useful for converting non-petroleum based bio-renewable feeds to low molecular weight, low boiling hydrocarbon products, e.g. gasoline.

For example, U.S. Patents Application Publication Nos. 2008/0035528 and 2007/0015947 disclose FCC processes for producing olefins from a bio-renewable feed source, e.g. vegetable oils and greases, or a feedstock containing a petroleum fraction and a fraction containing a bio-renewable feed source. The process involves first treating the bio-renewable feed source in a pretreatment zone at pretreatment conditions to remove contaminants present in the feed source and produce an effluent stream. The effluent from the pretreatment step is thereafter contacted with an FCC catalyst under FCC conditions to provide olefins. The FCC catalyst comprises a first component comprising a large pore zeolite, e.g. a Y-type zeolite, and a second component comprising a medium pore zeolite, ZSM-5 and the like, which components may or may not be present in the same matrix.

Japanese Unexamined Patent Application Publications 2007-177193, 2007-153924 and 2007-153925 disclose FCC processes for processing a stock oil containing a biomass. The processes involve first contacting stock oil containing a biomass with a catalyst that contains 10-50 mass % ultra-stable Y zeolite which may contain alkaline rare earth under FCC conditions and thereafter regenerating the catalyst in the regeneration zone to inhibit the amount of coke generated during the processing of the biomass.

There remains a need in the catalyst industry for improved processes for the conversion of feedstocks containing bio-renewable feed to produce lower molecular weight hydrocarbon products, e.g. gasoline.

### SUMMARY OF THE INVENTION

It has now been discovered that the use of certain rare earth-containing zeolite based fluid catalytic cracking (FCC) catalyst provides improved catalytically cracking of a feedstock containing at least one bio-renewable feed during a FCC process. Unexpectedly, it has been found that a Y-type zeolite based FCC catalyst containing at least 1 wt % rare earth and having a high zeolite surface area to matrix surface area ratio provides improved coke to bottoms selectivity during the catalytic conversion of feeds comprising at least one bio-renewable feed fraction to lower molecular weight hydrocarbons during an FCC process. Advantageously, Y-type zeolite FCC catalysts having a high ratio of zeolite surface area to matrix surface area offer increased activity under FCC conditions to catalytically crack a feedstock containing at least one bio-renewable feed to lower molecular weight molecules and provides increased bottoms conversion at constant coke formation as compared to bottoms conversion and coke formation obtainable using conventional Y-type zeolite based FCC catalysts.

In accordance with the process of the invention, a feedstock comprising at least one bio-renewable feed fraction is contacted under FCC conditions with catalytic cracking catalyst comprising a microporous zeolite having catalytic cracking ability under FCC conditions, a mesoporous matrix, and at least 1 wt % (based on the total weight of the catalyst) of a rare earth metal oxide, said catalyst having a zeolite surface area-to-matrix surface area ratio, as represented by Z/M ratio, of at least 2, to obtain a cracked product. In a preferred embodiment of the invention, the Z/M ratio of the cracking catalyst is greater than 2. Preferably, the catalyst comprise a Y-type zeolite, most preferably a rare earth exchanged Y-type zeolite having greater than 1 wt % of a rare-earth metal oxide, based on the total weight of the catalyst, in a matrix material having pores in the mesopore range. Preferably, the feedstock is a blend of a hydrocarbon feedstock and at least one bio-renewable feed.

Accordingly, it is an advantage of the present invention to provide simple and economical process for catalytically converting a feedstock containing at least one bio-renewable feed fraction to produce lower molecular weight hydrocarbon products.

It is also an advantage of the present invention to provide an improved FCC process for catalytically converting a feedstock containing at least one bio-renewable feed fraction, to produce lower molecular weight hydrocarbon products.

It is another advantage of the present invention to provide an improved FCC process for catalytic cracking feedstocks comprising a blend of at least one hydrocarbon feed and at least one bio-renewable feed, to produce lower molecular weight hydrocarbon products.

It is a further advantage of the present invention to provide an FCC process for catalytic cracking a feedstock comprising at least one bio-renewable which process offers increased conversion and yields as compared to conventional FCC processes.

It is also an advantage of the present invention to provide an FCC process for catalytic cracking a feedstock comprising at least on bio-renewable feed fraction, which process offers improved bottoms conversion at constant coke formation during an FCC cracking process as compared to conventional FCC processes.

These and other aspects of the present invention are described in further details below.

### BRIEF DESCRIPTION OF THE DRAWINGS

FIG. 1 is a graphic representation of the comparison of the bottoms yield (wt %) versus coke yield (wt %) obtained by ACE testing a feed containing a blend of 15% palm oil and 85% of a VGO/resid hydrocarbon blend using a high zeolite surface area-to-matrix surface area ratio catalyst (Catalyst A) and a low zeolite surface area-to-matrix surface area ratio catalyst (Catalyst B).

FIG. 2 is a graphic representation of the comparison of the catalyst-to-oil ratio versus conversion (wt %) obtained from the catalytic cracking of a feed containing a blend of 15% palm oil and 85% of a VGO/resid hydrocarbon blend using a high zeolite surface area-to-matrix surface area ratio catalyst in accordance with the invention and a low zeolite surface area-to-matrix surface area ratio catalyst.

FIG. 3 is a graphic representation of the comparison of the bottoms yield (wt %) versus coke yield (wt %) obtained from the catalytic cracking of a feed containing a blend of 15% soy oil and 85% of a VGO/resid hydrocarbon blend using a high zeolite surface area-to-matrix surface area ratio catalyst in accordance with the invention and a low zeolite surface area-to-matrix surface area catalyst.

FIG. 4 is a graphic representation of the comparison of the catalyst-to-oil ratio versus conversion (wt %) obtained from the catalytic cracking of a feed containing a blend of 15% soy oil and 85% of a VGO/resid hydrocarbon blend using a high zeolite surface area-to-matrix surface area catalyst in accordance with the invention and a low zeolite surface area-to-matrix surface area ratio catalyst.

FIG. 5 is a graphic representation of the comparison of the bottoms yield (wt %) versus coke yield (wt %) obtained from the catalytic cracking of a feed containing a blend of 15% rapeseed oil and 85% of a VGO/resid hydrocarbon blend using a high zeolite surface area-to-matrix surface area ratio catalyst in accordance with the invention and a low zeolite surface area-to-matrix surface area ratio catalyst.

FIG. 6 is a graphic representation of the comparison of the catalyst-to-oil ratio versus conversion (wt %) obtained from the catalytic cracking of a feed containing a blend of 15% rapeseed oil and 85% of a VGO/resid hydrocarbon blend using a high zeolite surface area-to-matrix surface area ratio catalyst in accordance with the invention and a low zeolite surface area-to-matrix surface area catalyst.

### DETAILED DESCRIPTION OF THE INVENTION

In accordance with the process of the present invention, a feedstock having at least one bio-renewable feed fraction is contacted under fluid catalytic cracking (FCC) conditions with a circulating inventory of catalytic cracking catalyst comprising primarily a zeolite, matrix and a rare-earth metal oxide and possessing a zeolite surface area to matrix surface area ratio, as represented by Z/M ratio, of at least 2.

In a preferred embodiment of the invention the process comprises obtaining a blended feedstock of a bio-renewable feed and a petroleum based hydrocarbon feed; providing a fluid catalytic cracking catalyst comprising a microporous, zeolite component having catalytic cracking activity under fluid catalytic cracking condition, a mesoporous matrix and at least 1 wt % rare earth metal oxide, based on the total weight of the catalyst, wherein the catalyst possess a Z/M ratio of at least 2; and contacting the blended feedstock with the catalytic cracking catalyst under FCC conditions to obtain cracked products.

For purposes of this invention the term “bio-renewable” or “bio-feed” is herein interchangeably, to designate any feed or fraction of a feed or feedstock that has a fat component derived from plant or animal oil. Typically, the feed or fraction comprises primarily triglycerides and free fatty acids (FFA). The tri-glycerides and FFAs contain aliphatic hydrocarbon chains in their structure having 14 to 22 carbons. Examples of such feedstocks include, but are not limited, canola oil, corn oil, soy oils, rapeseed oil, soybean oil, palm oil, colza oil, sunflower oil, hempseed oil, olive oil, linseed oil, coconut oil, castor oil, peanut oil, mustard oil, cotton seed oil, inedible tallow, inedible oil, e.g. jatropha oil, yellow and brown greases, lard, train oil, fats in milk, fish oil, algal oil, tall oil, sewage sludge and the like. Another example of a bio-renewable feedstock that can be used in the present invention is tall oil. Tall oil is a by-product of the wood processing industry. Tall oil contains esters and rosin acids in addition to FFAs. Rosin acids are cyclic carboxylic acids. The triglycerides and FFAs of the typical vegetable or animal fat contain aliphatic hydrocarbon chains in their structure which have about 8 to about 24 carbon atoms. Pyrolysis oils, which are formed by the pyrolysis of cellulosic waste material, can also be used as a non-petroleum feedstock or a portion or fraction of the feedstock.

For purposes of this invention, the phrase “fluid catalytic cracking conditions” or “FCC conditions” is used herein to indicate the conditions of a typical fluid catalytic cracking process, wherein a circulating inventory of a fluidized cracking catalyst is contacted with a heavy feedstock, e.g. hydrocarbon feedstock, bio-renewable feedstock, or a mixture thereof, at elevated temperature to convert the feedstocks into lower molecular weight compounds.

The term “fluid catalytic cracking activity” is used herein to indicate the ability of a compound to catalyze the conversion of hydrocarbons and/or fat molecules to lower molecular weight compounds under fluid catalytic cracking conditions.

For purposes of this invention, the term “matrix” is used herein to indicate all mesoporous materials, i.e. materials having pores with a pore radii of at least 20 Angstroms as measured by BET t-plot (see Johnson, J. M. F. L., J. Cat 52, pgs 425-431 (1978)), comprising the catalytic cracking catalyst of the invention, including any binders and/or fillers, e.g. clay and the like, and excluding the catalytically active zeolite which typically will have pores in the micropore range, i.e., openings less than 20 Angstroms as measured by BET t-plot.

Feedstocks useful in the present invention comprise petroleum based hydrocarbon feedstocks comprising at least one bio-renewable feed fraction. Petroleum based hydrocarbons feedstocks useful in the present invention typically include, in whole or in part, a gas oil (e.g., light, medium, or heavy gas oil) having an initial boiling point above about 120° C., a 50% point of at least about 315° C., and an end point up to about 850° C. The feedstock may also include deep cut gas oil, vacuum gas oil (VGO), thermal oil, residual oil, cycle stock, whole top crude, tar sand oil, shale oil, synthetic fuel, heavy hydrocarbon fractions derived from the destructive hydrogenation of coal, tar, pitches, asphalts, hydrotreated feedstocks derived from any of the foregoing, and the like. As will be recognized, the distillation of higher boiling petroleum fractions above about 400° C. must be carried out under vacuum in order to avoid thermal cracking. The boiling temperatures utilized herein are expressed in terms of convenience of the boiling point corrected to atmospheric pressure. Even high metal content resids or deeper cut gas oils having an end point of up to about 850° C. can be cracked using the invention.

In one embodiment of the invention, the feedstock is a blended feedstock, i.e. feedstocks comprising both hydrocarbon feed and bio-renewable feed fractions. Blended feedstocks useful in the process of the invention typically comprise from about 99 to about 25 wt % hydrocarbon feedstock and from about 1 to about 75 wt % bio-renewable feedstocks. Preferably, the blended feedstock comprises from about 97 to about 80 wt % hydrocarbon feedstock and from about 3 to about 20 wt % of a bio-renewable feedstock.

Zeolite based fluid catalytic cracking catalyst useful in the present invention may comprise any zeolite that has catalytic cracking activity under fluid catalytic cracking conditions. Preferably, the zeolite component is a synthetic faujasite zeolite, such as a USY or a rare earth exchanged USY faujasite zeolite. The zeolite may also be exchanged with a combination of metal and ammonium and/or acid ions. It is also contemplated that the zeolite component may comprise a mixture of zeolites such as synthetic faujasite in combination with mordenite, Beta zeolites and ZSM type zeolites. Generally, the zeolite cracking component comprises from about 10 to about 60 wt % of the cracking catalyst. Preferably, the zeolite cracking component comprises from about 20 to about 55 wt %, most preferably, from about 30 wt % to about 50 wt %, of the catalyst composition.

Suitable matrix materials useful to prepare high Z/M ratio catalyst compositions useful in the present invention include silica, alumina, silica alumina, binders and optionally clay. Suitable binders include alumina sol, silica sol, aluminum phosphate and mixtures thereof. Preferably, the binder is an alumina binder selected from the group consisting of an acid peptized alumina, a base peptized alumina and aluminum chlorhydrol.

The matrix material may be present in the invention catalyst in an amount of up to about 90 wt % of the total catalyst composition. In a preferred embodiment of the invention, the matrix is present in an amount ranging from about 40 to about 90 wt %, most preferably, from about 50 to about 70 wt %, of the total catalyst composition.

Matrix materials useful in the present invention may also optionally contain clay. While kaolin is the preferred clay component, it also contemplated that other clays, such as modified kaolin (e.g. metakaolin) may be optionally included. When used, the clay component will typically comprise from about 0 to about 70 wt %, preferably about 25 to about 60 wt % of the catalyst composition.

In accordance with the present invention, catalyst compositions useful in the invention process will posses a pore system comprising pores in the micropore and the mesopore range. Typically, catalyst compositions useful in the present invention comprise a high zeolite surface area to matrix surface area ratio. For purposes of the invention, the term “matrix surface area” is used herein to indicate the surface area attributable to the matrix material comprising the catalyst, which material will generally have a pore size of 20 Angstroms or greater as measured by BET t-plot The term “zeolite surface area” is used herein to indicate the surface area attributable to the fluid catalytically active zeolite comprising the catalyst, which zeolite will typically have a pore size of less than 20 Angstroms as measured by BET t-plot. In accordance with the present invention, the catalyst composition typically comprises a Z/M ratio of at least 2. In a preferred embodiment of the invention, the catalyst comprises a Z/M ratio of greater than 2. Generally, the Z/M ratio of catalysts compositions useful in the present invention ranges from about 2 to about 15, preferably from about 3 to about 10.

High Z/M ratio catalyst compositions useful in the present invention also comprises at least 1 wt % rare earth metal oxide based on the total weight of the catalyst. Preferably, the catalysts comprise from about 1 to about 10, most preferably, from about 1.5 to about 5, wt % rare earth metal oxide based on the total weight of the catalyst. The rare earth metal oxide may be present in the catalyst as an ion exchanged into the zeolite component, or alternatively, may be incorporated into the matrix as rare earth oxide or rare earth oxychloride. The rare earth metal oxide may also be incorporated into the catalyst as a component during manufacture of the catalyst. It is also within the scope of the present invention that the rare earth may be impregnated on the surface of the catalyst following manufacture of the catalyst composition. Suitable rare earth metals include, but are not limited to, elements selected from the group consisting of elements of the Lanthanide Series having an atomic number of 57-71, yttrium and mixtures thereof. Preferably, the rare earth metal is selected from the group consisting of lanthum, cerium and mixtures thereof.

Catalyst compositions useful in the present invention will typically have a mean particle size of about 40 to about 150 μm, more preferably from about 60 to about 90 μm. Typically, the catalyst compositions of the invention will possess a Davison Index (DI) sufficient to maintain the structural integrity of the compositions during the FCC process. Typically a DI value of less than 30, more preferably less than 25 and most preferably less than 20, will be sufficient.

Suitable high Z/M ratio catalyst compositions useful in the present invention include, but are not limited to, catalyst compositions currently being made and sold by W.R. Grace & Co.-Conn under the tradename, IMPACT®. Alternatively, suitable catalyst compositions in accordance with the invention may be prepared by forming an aqueous slurry containing an amount of zeolite, matrix material and optionally clay sufficient to provide from about 10 to about 60 wt % of zeolite component, about 40 to about 90 wt % of the matrix material and about 0 to about 70 wt % of clay in the final catalyst. The aqueous slurry is milled to obtain a homogeneous or substantially homogeneous slurry, i.e. a slurry wherein all the solid components of the slurry have an average particle size of less than 10 μm. Alternatively, the components forming the slurry are milled prior to forming the slurry. The aqueous slurry is thereafter mixed to obtain a homogeneous or substantially homogeneous aqueous slurry.

The aqueous slurry is thereafter subjected to a spraying step using conventional spray drying techniques. During the spray drying step, the slurry is converted into solid catalyst particles that comprise zeolite and the matrix material including binder and optionally fillers. The spray dried catalyst particles typically have an average particle size on the order of about 50 to about 70 p.m.

Following spray drying, the catalyst particles are calcined at temperatures ranging from about 370° C. to about 760° C. for a period of about 20 minutes to about 2 hours. Preferably, the catalyst particles are calcined at a temperature of about 600° C. for about 45 minutes. The catalyst particles may thereafter be optionally ion exchanged and/or washed, preferably with water, to remove excess alkali metal oxide and any other soluble impurities. The washed catalyst particles are separated from the slurry by conventional techniques, e.g. filtration, and dried to lower the moisture content of the particles to a desired level, typically at temperatures ranging from about 100° C. to 300° C.

It is further within the scope of the present invention that high Z/M ratio catalyst compositions in accordance with the invention may be used in combination with other additives conventionally used in a catalytic cracking process, e.g. SOₓ reduction additives, NOₓ reduction additives, gasoline sulfur reduction additives, CO combustion promoters, additives for the production of light olefins which may contain ZSM-5, and the like.

In accordance with the process of present invention, fluid catalytic cracking of a hydrocarbon bio-feed or a feedstock having a relatively high molecular weight hydrocarbon fraction and a bio-feed fraction in the FCC unit results in the production of a hydrocarbon products of lower molecular weight, e.g. gasoline. The FCC unit useful in the present invention is not particularly restricted as long as the unit contains a reaction zone, a separation zone, a stripping zone and a regeneration zone. The significant steps of the FCC process typically comprises:
(i) catalytically cracking a bio-renewable feed containing feedstock in a catalytic cracking zone, normally a riser cracking zone, operating at catalytic cracking conditions by contacting feed with a source of hot, regenerated cracking catalyst to produce an effluent comprising cracked products and spent catalyst containing coke and strippable hydrocarbons;
(ii) discharging and separating the effluent, normally in one or more cyclones, into a vapor phase rich in cracked product and a solids rich phase comprising the spent catalyst;
(iii) removing the vapor phase as product and fractionating the product in the FCC main column and its associated side columns to form gas and liquid cracking products including gasoline;
(iv) stripping the spent catalyst, usually with steam, to remove occluded hydrocarbons from the catalyst, after which the stripped catalyst is oxidatively regenerated in a catalyst regeneration zone to produce hot, regenerated catalyst, which is then recycled to the cracking zone for cracking further quantities of feed.

Within the reaction zone of the FCC unit, the FCC process is typically conducted at reaction temperatures of about 480° C. to about 600° C. with catalyst regeneration temperatures of about 600° C. to about 800° C. As it is well known in the art, the catalyst regeneration zone may consist of a single or multiple reactor vessels.

A catalyst-oil-ratio of typically, about 3 to about 12, preferably, about 5 to about 10; a hydrocarbon partial pressure in the reactor of typically, 1 bar to about 4 bar, preferably about 1.75 bar to about 2.5 bar; and a contact time between the feedstock and the catalyst of 1 to 10 seconds, preferably 2 to 5 seconds. The term “catalyst-oil-ratio’ as used in the present invention refers to the ratio of the catalyst circulation amount (ton/h) and the feedstock supply rate (ton/h). The term “hydrocarbon partial pressure” is used herein to indicate the overall hydrocarbon partial pressure in the riser reactor. The term “catalyst contact time” is used herein to indicate the time from the point of contact between the feedstock and the catalyst at the catalyst inlet of the riser bed reactor until separation of the reaction products and the catalyst at the stripper outlet.

The outlet temperature of the reaction zone as used in the present invention refers to the outlet temperature of the fluidized riser reactor. Generally, the outlet temperature of the reaction zone in the present invention will range from about 480° C. to about 600° C. It is also within the scope of the present invention that the FCC unit may comprise any device conventionally used for processing bio-renewable feeds.

In accordance with the process of the invention, high Z/M ratio cracking catalyst compositions useful in the invention process may be added to a circulating FCC catalyst inventory while the cracking process is underway or they may be present in the inventory at the start-up of the FCC operation. The catalyst compositions may be added directly to the cracking zone or to the regeneration zone of the FCC cracking apparatus, or at any other suitable point in the FCC process.

As will be understood by one skilled in the arts, the amount of catalyst used in the cracking process will vary from unit to unit depending on such factors as the feedstock to be cracked, operating conditions of the FCCU and desired output. Preferably, the amount of the high Z/M ratio catalyst is an amount sufficient to provide increased conversion of fat and/or oil molecules as well as heavy hydrocarbon molecules to lower molecular weight hydrocarbons, while simultaneously increasing bottoms conversion at constant coke formation as compared to the conversion and bottoms conversion obtained during a conventional FCC process. Typically, the amount of the high Z/M ratio catalyst used is an amount sufficient to maintain a Z/M ratio of greater than 2 and at least 1 wt %, preferably from about 1 to about 10 wt %, of rare earth in the entire cracking catalyst inventory.

In accordance with the process of the invention, bio-renewable feeds containing animal and/or plant fats and/or oils alone or blended with any typical hydrocarbon feedstock are cracked to produce cracked products of low molecular weight. The process is particularly useful for the production of transportations fuels, e.g. gasoline, diesel fuel. Very significant increases, i.e. about 10% to about 20%, in bottoms conversion at constant coke production are achievable using the process of the invention when compared to the use of conventional zeolite based FCC catalyst compositions having a low Z/M ratio. However, as will be understood by one skilled in the arts, the extent of bottoms conversion will depend on such factors as reactor temperature, catalyst to oil ratio and feedstock type. Advantageously, the process of the invention provides an increase in bottom cracking at constant coke production during the FCC process as compared to the use of conventional zeolite based FCC catalyst compositions having a low Z/M ratio.

To further illustrate the present invention and the advantages thereof, the following specific examples are given. The examples are given as specific illustrations of the claimed invention. It should be understood, however, that the invention is not limited to the specific details set forth in the examples.

All parts and percentages in the examples as well as the remainder of the specification that refers to compositions or concentrations are by weight unless otherwise specified.

Further, any range of numbers recited in the specification or claims, such as that representing a particular set of properties, units of measure, conditions, physical states or percentages, is intended to literally incorporate expressly herein by reference or otherwise, any number falling within such range, including any subset of numbers within any range so recited.

### EXAMPLES

Blended feedstocks in the Examples below were catalytically cracked using an Advanced Catalyst Evaluation (ACE) unit, as described in U.S. Pat. No. 6,069,012, using a commercially available high Z/M ratio catalyst, IMPACT®-1495, obtained from Davison Refining Technologies of W.R. Grace & Co., (Catalyst A) and a commercially available low Z/M ratio catalyst MIDAS®-138 currently being sold by Davison Refining Technologies of W.R. Grace & Co., (Catalyst B), respectively. Table 1 displays the microporous (zeolite) and mesoporous (matrix) surface areas as measured by BET t-plot (Johnson, M. F. L. P., J. Cat 52, pgs 425-431 (1978)) for both fresh and steam deactivated catalysts. The steam deactivated samples were steamed using the cyclic propylene steam (see Lori T. Boock, Thomas F. Petti, and John A. Rudesill, ACS Symposium Series, 634, 1996, 171-183) Catalyst A had respective Z/M ratios of 5.3 and 4.2 for the fresh and steamed catalyst, while Catalyst B had respective Z/M ratios of 1.4 and 1.3 for the fresh and steamed catalyst.

TABLE 1

| Properties | Catalyst A | Catalyst B |
|---|---|---|
| Fresh Microporous surface area, m²/g | 267 | 163 |
| Fresh Mesoporous surface area, m²/g | 50 | 114 |
| Ratio Microporous to Mesoporous | 5.3 | 1.4 |
| *Steamed Microporous surface area, m²/g | 152 | 99 |
| *Steamed Mesoporous surface area, m²/g | 36 | 76 |
| *Ratio steamed microporous to steamed | 4.2 | 1.3 |
| mesoporous | | |
| Unit Cell, Å | 24.53 | 24.53 |
| Pore Volume (cc/g) | 0.36 | 0.46 |
| Al₂O₃, wt % | 46.7 | 51.3 |
| Re₂O₃, wt % | 5.1 | 2.1 |

*Deactivated by cyclic propylene steam with 1000 ppm Nickel and 2000 ppm Vanadium.

### Example 1

A vacuum gas oil (VGO) and resid blended hydrocarbon feedstock was blended with a palm oil to provide a hydrocarbon feedstock having 85% VGO and resid blend and 15% palm oil. The properties of the VGO/resid blend and the palm oil are recorded in Table 2 below:

TABLE 2

| | VGO/resid | |
|---|---|---|
| | blend | Palm Oil |
| API (°) | 24.4 | 22.98 |
| Distillation, ° F. | | |
| IBP | 494 | 625 |
| 10 | 689 | 1026 |
| 30 | 775 | 1062 |
| 50 | 834 | 1079 |
| 70 | 899 | 1090 |
| 90 | 1018 | 1146 |
| 95 | 1110 | 1197 |
| FBP | 1279 | 1302 |
| Sulfur, ppm | 5300 | 1 |
| Nitrogen, ppm | 813 | 2 |

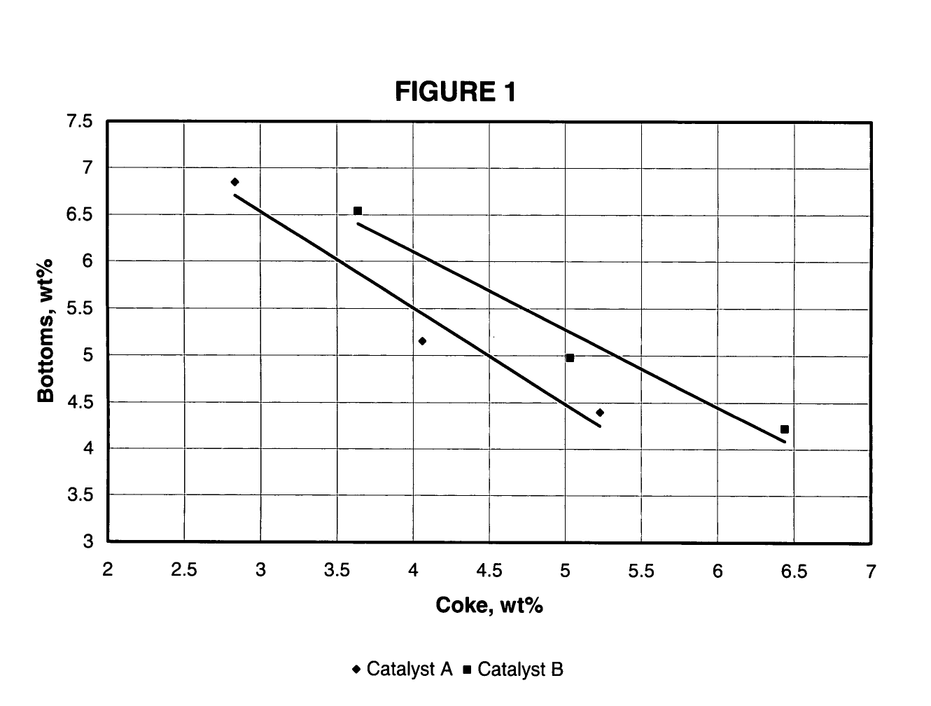

The blended palm oil/hydrocarbon feedstock was catalytically cracked using an ACE unit using Catalyst A and Catalyst B as described herein above. As shown in FIG. 1 below, the high Z/M ratio catalyst, Catalyst A, exhibited superior performance for bottoms conversion at constant coke when compared to the performance of the low Z/M ratio catalyst, Catalyst B. Clearly, the coke and bottoms yields for the high Z/M ratio catalyst (Catalyst A) were lower than those obtained using low Z/M ratio catalyst (Catalyst B).

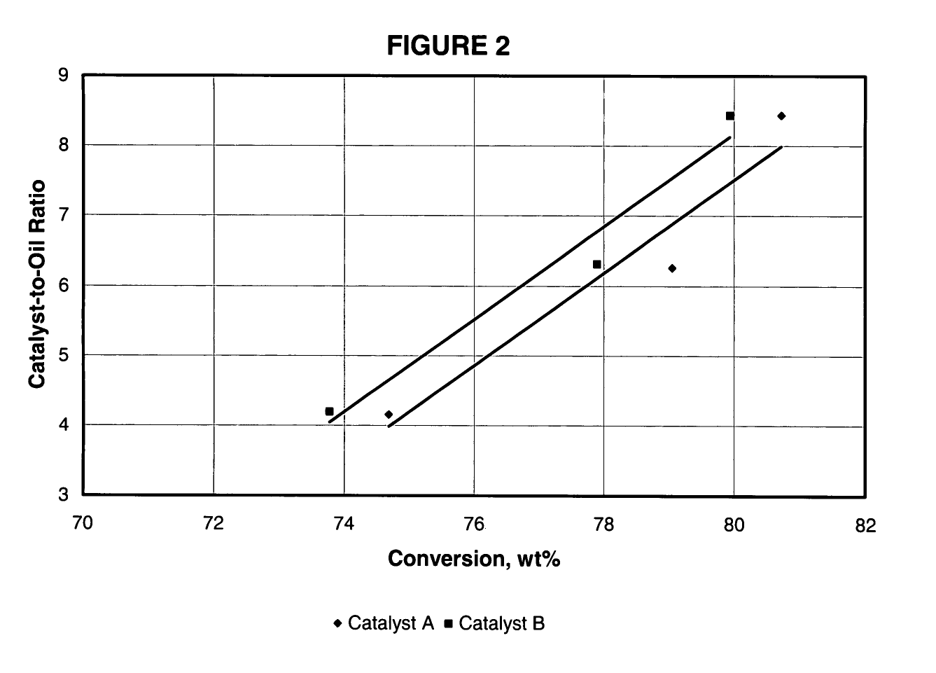

Further, as shown in FIG. 2, a comparison of the catalyst-to-oil ratio and the weight percentage of conversion, with a conversion defined as 100% minus the weight % of liquid products that boil over 221° C., obtained for Catalyst A and Catalyst B, showed that the same conversion is achieved at lower catalyst-to-oil ratio for Catalyst A than for Catalyst B. This indicates an increased activity to convert a hydrocarbon feedstock containing at least one bio-renewable fraction using a high Z/M ratio catalyst in accordance with the invention when compared to the activity obtainable using a low Z/M ratio catalyst.

### Example 2

A vacuum gas oil (VGO) and resid blended hydrocarbon feedstock was blended with a soy oil to provide a hydrocarbon feedstock having 85% VGO and resid blend and 15% soy oil. The properties of the VGO/resid blend and the soy oil are recorded in Table 3 below:

TABLE 3

| | VGO/resid | |
|---|---|---|
| | blend | Soy Oil |
| API (°) | 24.4 | 21.58 |
| Distillation, ° F. | | |
| IBP | 494 | 702 |
| 10 | 689 | 1069 |
| 30 | 775 | 1090 |
| 50 | 834 | 1102 |
| 70 | 899 | 1111 |
| 90 | 1018 | 1183 |
| 95 | 1110 | 1232 |
| FBP | 1279 | 1301 |
| Sulfur, ppm | 5300 | 0 |
| Nitrogen, ppm | 813 | 4 |

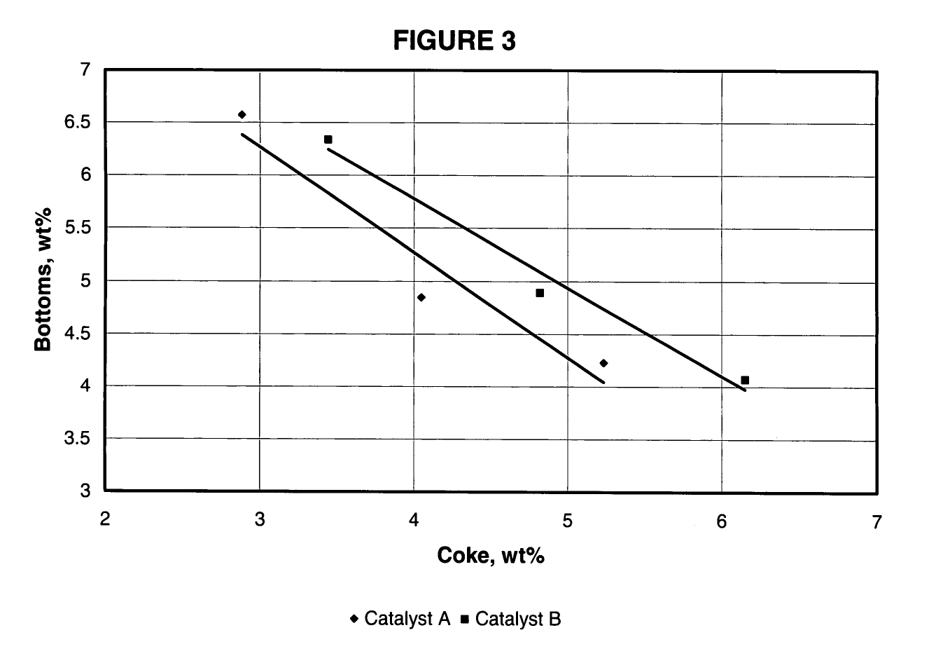

The blended soy oil/hydrocarbon feedstock was catalytically cracked using an ACE unit using Catalyst A and Catalyst B as described herein above. As shown in FIG. 3 below, the high Z/M ratio catalyst, Catalyst A, exhibited superior performance for bottoms conversion at constant coke when compared to the performance of the low Z/M ratio catalyst, Catalyst B. Clearly, the coke and bottoms yields for the high Z/M ratio catalyst (Catalyst A) were lower than those obtained using low Z/M ratio catalyst (Catalyst B).

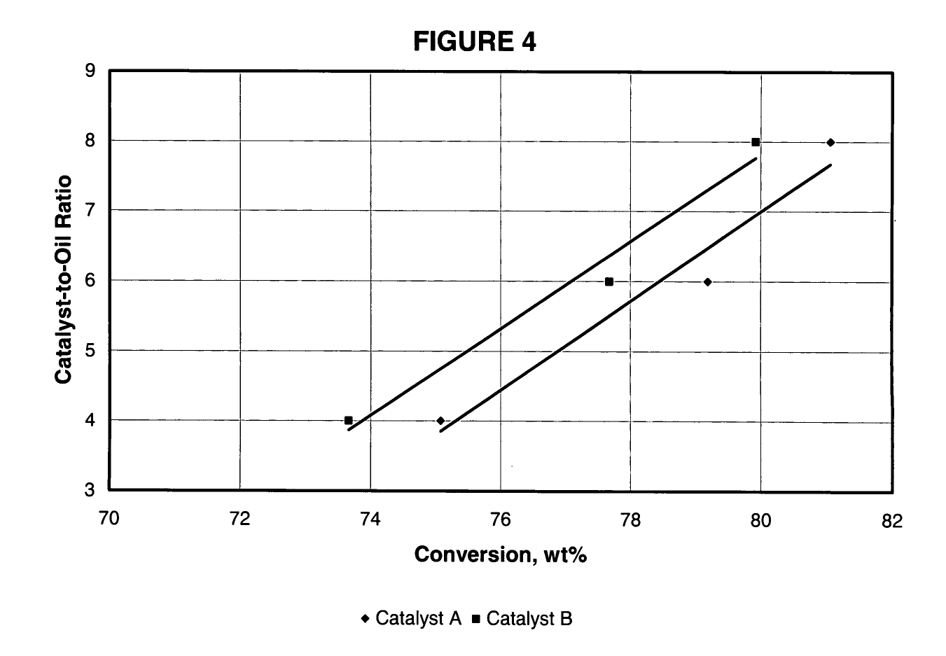

Further, as shown in FIG. 4, a comparison of the catalyst-to-oil ratio and the weight percentage of conversion, with a conversion defined as 100% minus the weight % of liquid products that boil over 221° C., obtained for Catalyst A and Catalyst B, showed that the same conversion is achieved at lower catalyst-to-oil ratio for Catalyst A than for Catalyst B. This indicates an increased activity to convert a hydrocarbon feedstock containing at least one bio-renewable fraction using a high Z/M ratio catalyst in accordance with the invention when compared to the activity obtainable using a low Z/M ratio catalyst.

### Example 3

A vacuum gas oil (VGO) and resid blended hydrocarbon feedstock was blended with a rapeseed oil to provide a hydrocarbon feedstock having 85% VGO and resid blend and 15% rapeseed oil. The properties of the VGO/resid blend and the rapeseed oil are recorded in Table 4 below:

TABLE 4

| | VGO/resid | |
|---|---|---|
| | blend | Rapeseed Oil |
| API (°) | 24.4 | 21.98 |
| Distillation, ° F. | | |
| IBP | 494 | 710 |
| 10 | 689 | 1077 |
| 30 | 775 | 1095 |
| 50 | 834 | 1106 |
| 70 | 899 | 1115 |
| 90 | 1018 | 1188 |
| 95 | 1110 | 1238 |
| FBP | 1279 | 1311 |
| Sulfur, ppm | 5300 | 3 |
| Nitrogen, ppm | 813 | 16 |

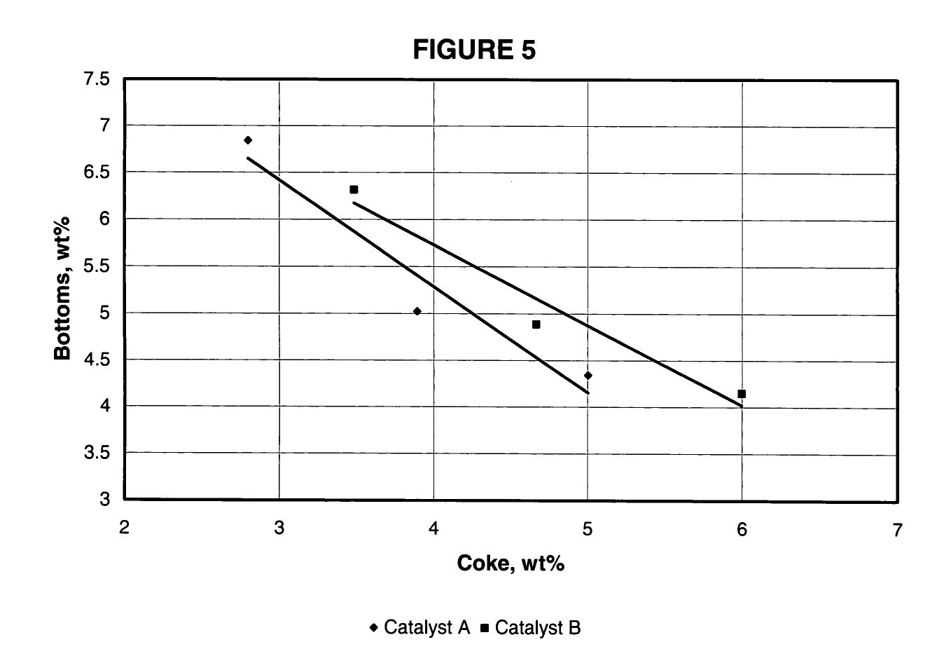

The blended rapeseed oil/hydrocarbon feedstock was catalytically cracked sing an ACE unit using Catalyst A and Catalyst B as described herein above. As shown in FIG. 5 below, the high Z/M ratio catalyst, Catalyst A, exhibited superior performance for bottoms conversion at constant coke when compared to the performance of the low Z/M ratio catalyst, Catalyst B. Clearly, the coke and bottoms yields for the high Z/M ratio catalyst (Catalyst A) were lower than those obtained using low Z/M ratio catalyst (Catalyst B).

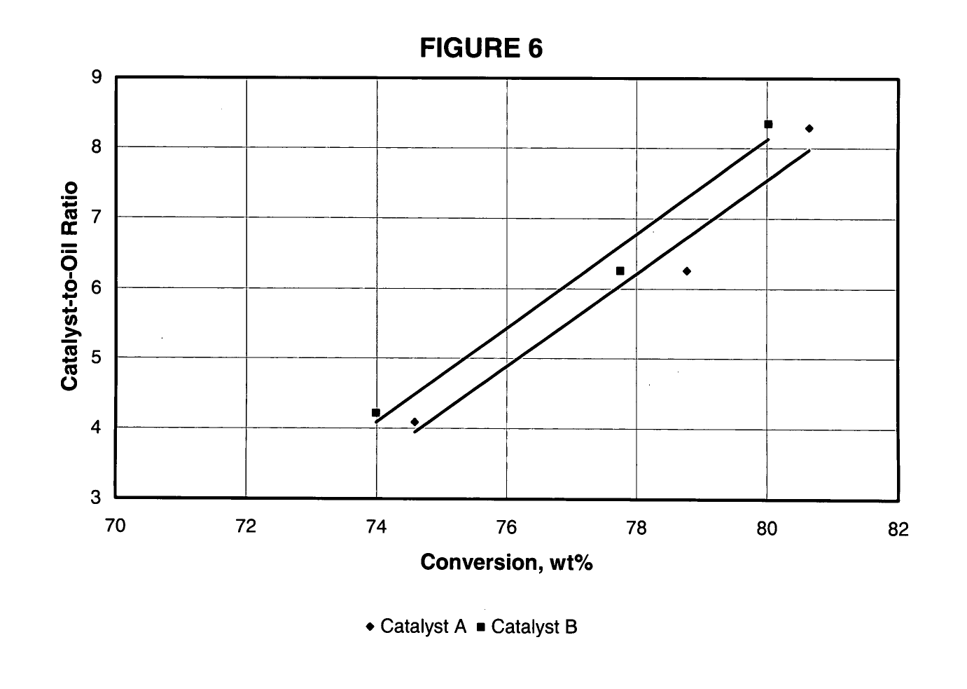

Further, as shown in FIG. 6, a comparison of the catalyst-to-oil ratio and the weight percentage of conversion, with a conversion defined as 100% minus the weight % of liquid products that boil over 221° C., obtained for Catalyst A and Catalyst B, showed that the same conversion is achieved at lower catalyst-to-oil ratio for Catalyst A than for Catalyst B. This indicates an increased activity to convert a hydrocarbon feedstock containing at least one bio-renewable fraction using a high Z/M ratio catalyst in accordance with the invention when compared to the activity obtainable using a low Z/M ratio catalyst.

What is claimed:

1. A process for the fluid catalytic cracking (FCC) of a feedstock comprising at least one bio-renewable feed, the process comprising
contacting a feedstock with at least one hydrocarbon fraction and at least one bio-renewable feed with catalytic cracking catalyst under FCC cracking conditions, wherein said catalyst comprises a zeolite having catalytic cracking activity, a matrix, and at least 1 wt %, based on the total weight of the catalyst, of a rare earth metal oxide, said catalyst having a zeolite surface area-to-matrix surface area ratio of at least 2; and
providing a cracked hydrocarbon product.

2. The process of claim 1 wherein the zeolite is a faujasite Y zeolite.

3. The process of claim 1 wherein the matrix is selected from the group consisting of silica, alumina, silica alumina and mixtures thereof.

4. The process of claim 1 wherein the hydrocarbon fraction comprises a petroleum based feedstock.

5. The process of claim 1 wherein the hydrocarbon fraction is a petroleum based feedstock selected from the group consisting of deep cut gas oil, vacuum gas oil (VGO), thermal oil, residual oil, cycle stock, whole top crude, tar sand oil, shale oil, synthetic fuel, heavy hydrocarbon fractions derived from the destructive hydrogenation of coal, tar, pitches, asphalts, hydrotreated feedstocks and mixtures thereof.

6. The process of claim 1, 4 or 5 wherein the bio-renewable fraction is a feedstock selected from the group consisting of canola oil, corn oil, soy oils, rapeseed oil, soybean oil, palm oil, colza oil, sunflower oil, hempseed oil, olive oil, linseed oil, coconut oil, castor oil, peanut oil, mustard oil, cotton seed oil, inedible tallow, inedible oil, yellow, brown greases, lard, train oil, fats in milk, fish oil, algal oil, tall oil, sewage sludge, tall oil and mixtures thereof.

7. The process of claim 6 wherein the inedible oil is jatropha oil.

8. The process of claim 1 wherein the zeolite surface area-to-matrix surface area is greater than 2.

9. The process of claim 1 or 8 wherein the surface area of the zeolite comprising the catalytic cracking catalyst is less than 20 Angstroms as measured by BET t-plot.

10. The process of claim 1 or 8 wherein the surface area of the matrix comprising the catalytic cracking catalyst is greater than 20 Angstroms as measured by BET t-plot.

11. The process of claim 1 wherein the rare earth metal oxide is an oxide of a metal selected from the group consisting of elements of the Lanthanide Series having an atomic number of 57-71, yttrium and mixtures thereof.

12. The process of claim 11 wherein the rare earth metal is selected from the group consisting of lanthum, cerium and mixtures thereof.

13. The process of claim 1 wherein the rare earth metal oxide is present in the catalytic cracking catalyst in an amount ranging from about 1 to about 10 wt % based on the total weight of the catalyst.

14. The process of claim 3 wherein the matrix further comprises clay.

15. The process of claim 3 or 14 wherein the matrix further comprises a binder.

16. The process of claim 15 wherein the binder is selected from the group consisting of alumina sol, silica sol, aluminum phosphate and mixtures thereof.

17. The process of claim 16 wherein the binder is an alumina sol selected from the group consisting of an acid peptized alumina, a base peptized alumina, aluminum chlorhydrol and mixtures thereof.
